(19) United States
(12) Patent Application Publication
(10) Pub. No.: US 2006/0094926 A1
(43) Pub. Date: May 4, 2006

(54) Multi-material penis constriction device

(76) Inventor: Peter Forsell, Zug (CH)

Correspondence Address: NIXON & VANDERHYE, PC, 901 NORTH GLEBE ROAD, 11TH FLOOR, ARLINGTON, VA, 22203, US

(21) Appl. No.: 10/522,538

(22) PCT Filed: Jun. 19, 2003
(86) PCT No.: PCT/SE03/01056

§ 371 Date: Aug. 19, 2005

Related U.S. Application Data
(60) Provisional application No. 60/398,809, filed on Jul. 29, 2002.

(51) Int. Cl. A61F 5/00

(52) U.S. Cl. 600/38

(57) ABSTRACT

An implantable constriction device (2) for constricting penile blood vessels of a patient for treating impotence comprises an elongate composite structure (84) adapted to constrict the exit penile veins or corpus cavernosa of the patient to restrict the penile venous blood flow. The elongate composite structure is composed of a base material (12), such as hard silicone, making the composite structure self-supporting. Property improving means (14) is provided for improving at least one physical property of the composite structure other than self-supporting properties, such as fatigue resistance, liquid impermeability, aggressive body cells resistance, anti-friction properties and lifetime.

The present invention relates to an implantable constriction device for constricting penile blood vessels of a patient, for treating impotence.

This kind of constriction device in the form of a banding device, in which a band encircles a portion of a patient's penile exit veins or corpus cavernosa to restrict the patient's penile venous blood flow has been used in surgery to treat impotence. In practice, the band is made of silicone, which is a material approved and widely used for implantation. Moreover, the silicone band has an acceptable tensile strength and is fairly resistant to aggressive body fluids. Where the band is hydraulically adjusted the hydraulic fluid used typically is an isotonic salt solution mixed with other conventional materials.

A problem of traditional silicone bands, however, is that the silicone material gives the band certain inadequate properties, such as poor fatigue resistance and poor endurance of static bending forces, which over time might result in breakage of the band. Furthermore, silicone is a material that is semi-permeable by liquid, which is a drawback to hydraulic silicone bands, because hydraulic fluid can escape by diffusing through the silicone material. As a result, accurate adjustments of a hydraulic band are difficult to perform because of loss of hydraulic fluid and the patient has to regularly visit a doctor for adding hydraulic fluid to and calibrating the constriction device. These inadequate properties are serious considering that the band should be implanted for the rest of the patient's life. Another problem is that the band somewhat restrains the dynamics movements of adjacent organs necessary for transportation of urine or fecal. As a consequence, the band might erode and over time injure the blood vessels or the corpus cavernosa.

The object of the present invention is to provide a new implantable constriction device for treating impotence having improved properties as compared to traditional constriction devices.

Accordingly, the present invention provides an implantable constriction device for constricting penile blood vessels of a patient, for treating impotence, characterised by an elongate composite structure adapted to constrict the exit penile veins or corpus cavernosa of the patient to restrict the penile venous blood flow, wherein the elongate composite structure is composed of a base material making the composite structure self-supporting and property improving means for improving at least one physical property of the composite structure other than self-supporting properties.

In accordance with a first embodiment of the invention, the property improving means comprises a coating coated on the base material at least along a side of the elongate composite structure that is intended to contact the penile veins or corpus cavernosa, wherein the coating has better aggressive body fluid resistance than the base material. Such a coating may comprise a Teflon™ or Parylene™ coating, or a biocompatible metal coating such as gold, silver or titanium. As a result, the constriction device can be protected from damaging influences of aggressive body fluids possibly for the rest of the patient's life.

Where the traditional silicone material constitutes the base material a Teflon™ or Parylene™ coating also provides the composite structure with better anti-friction properties than the base material. Good anti-friction properties of the composite structure reduce the risk of the elongate composite structure eroding the penile veins or corpus cavernosa. This is proven by tests, in which the surface of traditional silicone bands has been polished before use. Accordingly, the test results indicate significant improvements in avoiding erosion of the penile veins or corpus cavernosa.

Furthermore, the Teflon™, Parylene™ or metal coating also makes the composite structure, in which the base material is made of silicone, stronger than the traditional silicone band. A stronger band reduces the risk of fracture.

In one alternative of the first embodiment, the elongate composite structure is designed for mechanical adjustment, such as the mechanical solutions disclosed in WO 01/47434. In this alternative, the property improving means comprises a core of a soft viscoelastic material, such as silicone gel, typically having a hardness less than 20 Shure, cellulose gel or collagen gel. Where silicone gel is chosen it may be “Med 3-6300” manufactured by Nusil. Hard silicone constitutes the base material, typically having a hardness of at least 60 Shure, and covers the soft core of viscoelastic material. The soft core makes the implanted elongate composite structure less injurious to the penile veins or corpus cavernosa and reduces the injury of such organs. Furthermore, the soft core of viscoelastic material may be formed to enclose and protect mechanical adjustment components and other components of the composite structure, whereby fibrosis is prevented from growing into such components.

In another alternative of the first embodiment, the elongate composite structure is designed for hydraulic adjustment, such as the hydraulic solutions disclosed in WO 01/54626. In this alternative, the base material forms a closed tubing, which can be inflated by adding hydraulic fluid to the interior of the tubing and be deflated by withdrawing hydraulic fluid from the interior of the tubing. The coating of Teflon™, Parylene™ or metal may cover the inner surface of the tubing. The base material may form two coaxial tubular layers of hard silicon and the property improving means may comprise a tubular intermediate layer of a soft viscoelastic material located between the coaxial tubular layers. Alternatively, the base material may form an outer tubular layer, an inner arcuate layer attached to the outer tubular layer, the outer and inner layers defining a curved space extending longitudinally along the tubing. The property improving means may comprise a viscoelastic material filling the space. The tubing is applied around the penile veins or corpus cavernosa so that the space with viscoelastic material is located closest to the penile exit veins or corpus cavernosa. The viscoelastic material gives the advantages that erosion of the penile veins or corpus cavernosa is reduced and the risk of hydraulic fluid leaking from the tubing is decreased.

In accordance with a second embodiment of the invention, the base material forms a first layer and the property improving means comprises a second layer applied on the first layer, wherein the second layer is more fatigue resistant than the first layer. The first layer preferably comprises hard silicone, whereas the second layer preferably comprises a polyurethane layer. In a traditional silicone band, especially the tubular type, that is formed in a loop to constrict the penile veins or corpus cavernosa, the inner surface of the band loop that contacts the penile veins or corpus cavernosa forms bulges and creases that repeatedly changes as the band is subjected to dynamic movements from the penile veins or corpus cavernosa and when the size of the band is adjusted. As a consequence, the implanted traditional silicone band has the drawback that it may crack after some time due to fatigue of the silicon material. With the elongate composite structure of the invention, in which hard silicone may constitute the base material and a fatigue resistant polyurethane layer covers the silicone material on the side of the elongate composite structure that contacts the penile veins or corpus cavernosa, this drawback is eliminated.

The property improving means suitably comprises a coating that may be coated on the layer of hard silicone and/or the layer of polyurethane, wherein the coating has better aggressive body fluid resistance properties and/or better anti-friction properties than hard silicone. As described above in connection with the first embodiment the coating may comprise a Teflon™ or Parylene™ coating, or a biocompatible metal coating.

The layer of hard silicone may form an inflatable tubing and the layer of polyurethane may cover the hard silicone layer within the tubing.

In accordance with a third embodiment of the invention, the base material forms an inflatable tubing and the property improving means comprises a liquid impermeable coating coated on the base material. The coating may be coated on the external and/or internal surface of the tubing. Preferably, the liquid impermeable coating comprises a Parylene™ coating, or a biocompatible metal coating. Where hard silicone, which is a liquid semi-permeable material, constitutes the base material, the coating of Parylene™ or metal gives the advantage that the tubing may be inflated by hydraulic fluid under pressure without risking fluid diffusing through the silicone wall of the tubing.

Also in the third embodiment the base material may form two coaxial tubular layers of hard silicon and the property improving means may comprise a tubular intermediate layer of a soft viscoelastic material located between the coaxial tubular layers. Alternatively, the base material may form an outer tubular layer of hard silicone and an inner arcuate layer of silicone attached to the outer tubular layer. The outer and inner layers define a curved space extending longitudinally along the tubing and filled with the viscoelastic material. The tubing is intended to be applied around the penile veins or corpus cavernosa so that the space with viscoelastic material is located closest to the penile exit veins or corpus cavernosa.

In accordance with a fourth embodiment of the invention, the property improving means comprises gas, such as air, contained in a multiplicity of cavities formed in the base material to improve the flexibility of the composite structure. In this case, Teflon™ advantageously constitutes the base material. The cavities may be defined by net structures of the Teflon™ material. Thus, the resulted composite structure of Teflon™ and cavities with gas will be strong, flexible and aggressive body fluid resistant, and have good tensile strength and good anti-friction properties.

Also in the fourth embodiment the elongate composite structure may comprise an inflatable tubing.

The present invention also provides an implantable constriction device for constricting penile blood vessels of a patient, for treating impotence, the constriction device comprising an elongate composite structure adapted to constrict the exit penile veins or corpus cavernosa of the patient to restrict the penile venous blood flow, wherein the composite structure includes an elongate biocompatible self-supporting base material having surfaces exposed to aggressive body cells when the constriction device is implanted in the patient, and a cell barrier coating coated on the surfaces to prevent body cells from breaking down the base material, typically of silicone. If the base material were broken down by such body cells, typically macrophages or killer cells, histological particles would be spread in the human body.

The barrier coating may comprise a Parylene™ coating or a biocompatible metal coating.

Alternatively, the barrier coating may comprise a composite of different materials to achieve the cell-barrier protection as described above. There are several examples of such composite materials on the market, for example a composite of polyurethane and silicone called Elaston™.

The invention is explaied in more detail in the following with reference to the accompanying drawings, in which:

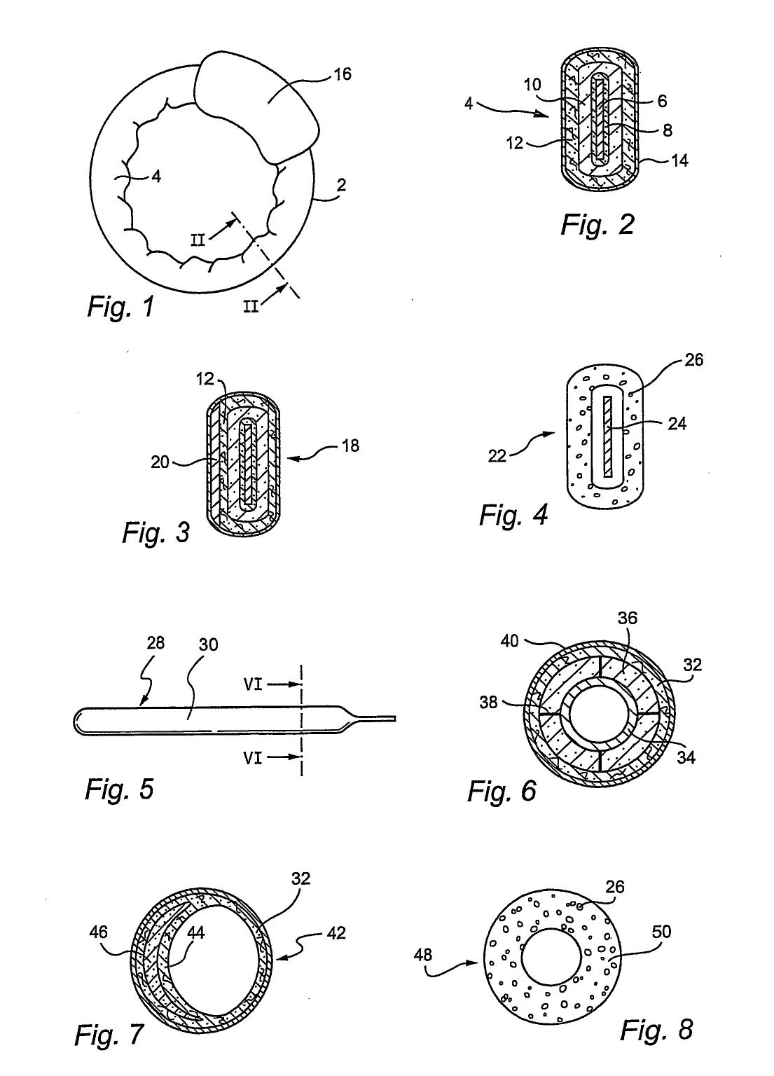

FIG. 1 is a front view of a mechanical constriction device according to the present invention,

FIG. 2 is an enlarged cross-section along the line II-II in FIG. 1,

FIGS. 3 and 4 are modifications of the embodiment shown in FIG. 2,

FIG. 5 is a front view of a hydraulic constriction device of the invention,

FIG. 6 is an enlarged cross-section along the line VI-VI in FIG. 5,

FIGS. 7-10 are modifications of the embodiment shown in FIG. 6, and

FIG. 11 is a modification of the embodiment shown in FIG. 2.

Referring to the drawing figures, like reference numerals designate identical or corresponding elements throughout the several figures.

FIG. 1 illustrates a mechanical constriction device 2 according to the invention comprising an elongate composite structure 4 adapted to extend around and constrict the exit penile veins or corpus cavernosa of a patient to restrict the penile venous blood flow therein. Referring to FIG. 2, the elongate composite structure 4 comprises a strong band 6 of nylon or the like, a tubular layer 8 of hard silicone, in which the band 6 slides, a soft layer 10 of a viscoelastic material, here a silicone gel having a hardness not more than 20 Shure, encircling the hard silicone layer 8, and a tubular layer 12 of a self-supporting base material of hard silicone having a hardness of at least 60 Shure, surrounding the soft silicon layer 10. A coating 14 of Teflon™, Parylene™ or a biocompatible metal, such as gold, silver or titanium, is coated on the outer hard silicone layer 12 to make the composite structure resistant to aggressive body fluids and to give the composite structure good anti-friction properties. A coating of Teflon™, Parylene™ or metal may also be coated on the internal surface of the inner tubular hard silicone layer 8 to reduce the friction between the nylon band 6 and the layer 8. The constriction device 2 has an adjustment means 16 that can displace the end portions of the nylon band 6 relative to each other to either increase or decrease the constriction of the penile veins or corpus cavernosa.

FIG. 3 shows an elongate composite structure 18 similar to that of FIG. 2, except that a layer 20 of a fatigue resistant material, here polyurethane, is applied on the hard silicone layer 12 along the inner side of the structure 18 that is intended to contact the exit penile veins or corpus cavernosa. Alternatively, the layer 20 may be tubular and surround the layer 12.

FIG. 4 shows a cross-section of an elongate composite structure 22 of an embodiment of the invention, in which Teflon™ constitutes the self-supporting base material, which is formed with a longitudinal cavity in which a strong nylon band 24 slides. Property improving means in the form of gas, here air, contained in a multiplicity of cavities 26 are formed in the base material to improve the flexibility thereof.

FIG. 5 shows a hydraulic constriction device 28 according to the invention comprising an elongate composite structure in the form of an inflatable tubing 30, in which the base material of hard silicone forms an outer tubular layer 32 and an inner coaxial layer 34. A viscoelastic material, here soft silicone gel, forms an intermediate layer 36 located between the tubular layers 32,34. Four longitudinal partition walls 38 between the tubular layers 32,34 divide the intermediate layer 36 into four sections to prevent the silicone gel from displacing in the circumferential direction of the tubing 30. (Also the embodiments according to FIGS. 2 and 3 may be provided with such longitudinal partition walls.) The outer layer 32 is coated with a coating 40 of Teflon™, Parylene™ or metal. Also the inner layer 34 may be coated with a coating of Teflon™, Parylene™ or metal. If a Parylene™ or metal coating is chosen the composite structure will be completely liquid impermeable.

FIG. 7 shows a tubing 42 similar to that of FIG. 6, except that an inner arcuate layer 44 is substituted for the inner tubular layer 34. The arcuate layer 44 is attached to the outer tubular layer 32, so that the outer tubular layer 32 and the arcuate layer 44 define a curved space extending longitudinally along the tubing 42. A viscoelastic material, here silicone gel 46, fills the space. In this embodiment there is no need for partition walls of the kind shown in the embodiment according to FIG. 6. The tubing 42 is intended to be applied around the exit penile veins or corpus cavernosa so that the space with the protecting soft silicone gel 46 is located close to the exit penile veins or corpus cavernosa.

As taught by the embodiment of FIG. 7, in the composite structures shown in FIGS. 2 and 3 the soft silicone gel may alternatively be applied in a longitudinal space close to the inner side of the elongate composite structure 4 and 18, respectively, that is intended to contact the exit penile veins or corpus cavernosa.

In the same manner as described above in connection with the embodiment of FIG. 3, a layer of a fatigue resistant material, here polyurethane, may be applied on the outer tubular layer 32 of hard silicone of the tubing 30 and 42, respectively, along the side of the tubing 30 and 42, respectively, that is intended to contact the exit penile veins or corpus cavernosa, when the tubing 30 and 42, respectively, encircles the exit penile veins or corpus cavernosa.

FIG. 8 shows a cross-section of an elongate composite structure 48 of an embodiment of the invention, in which Teflon™ constitutes the self-supporting base material, which is formed to an inflatable tubing 50. Property improving means in the form of gas contained in a multiplicity of cavities 26 are formed in the base material to improve the flexibility of the tubing 50.

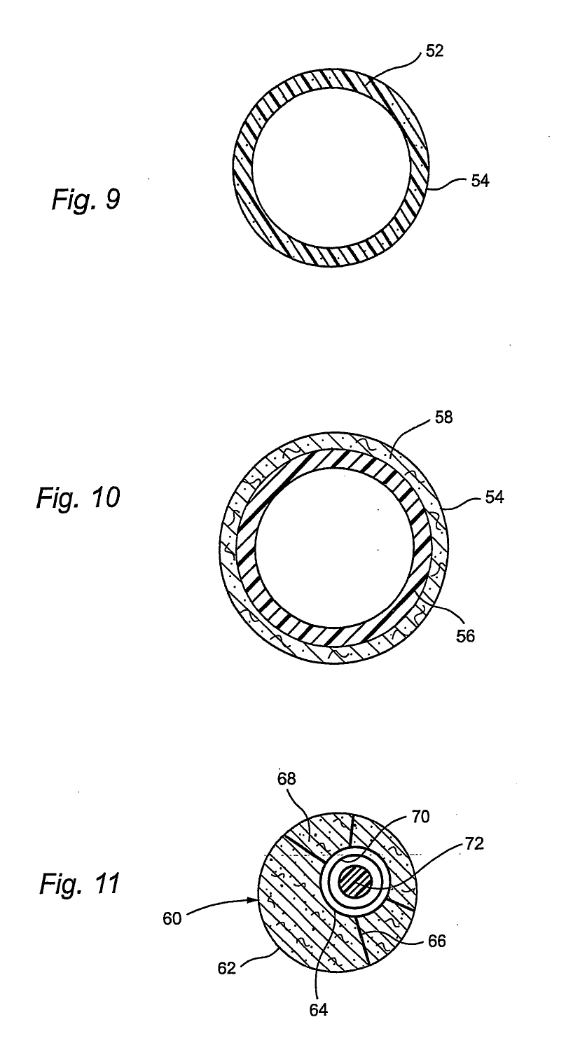

FIG. 9 shows a cross-section of a tubular composite structure of an embodiment of the invention, in which the self-supporting base material 52 is made of a polymer material suited for implantation, for example silicone or polyurethane. A property improving coating 54, for example made of Parylene™, Teflon™ or metal, is applied on the external surface or on both the external and internal surfaces of the tubular structure

FIG. 10 shows the same embodiment as FIG. 9 except that the base material comprises a layer 56 of polyurethane surrounded by a layer 58 of silicone.

FIG. 11 shows a cross-section of a mechanical constriction device of another embodiment of the invention, comprising a double walled tubing 60, an external wall 62 and an internal wall 64 spaced from the external wall 62, of a self-supporting base material of hard silicone. The tubing 60 has partition walls 66 dividing the space between the external and internal walls 62 and 64, respectively, of the tubing 60 into longitudinal cells 68, which are filled with a soft viscoelastic material, for example silicone gel. The internal wall 64 is coated with a friction reducing coating 70, for example made of Teflon™ or the like. A strong band 72 of nylon or the like slides in the tubing 60 on the friction reducing coating 70 to enable adjustment of the constriction device in the same manner as described above in connection with the embodiment according to FIGS. 1 and 2.

1-30. (canceled)

31. An implantable constriction device for constricting penile blood vessels of a patient for treating impotence, the constriction device comprising an elongate composite structure adapted to constrict the exit penile veins or corpus cavernosa of the patient to restrict the penile venous blood flow, wherein said elongate composite structure is composed of a base material making said composite structure self-supporting and property improving means for improving at least one physical property of said composite structure other than self-supporting properties.

32. An implantable constriction device according to claim 31, wherein said property improving means comprises a coating coated on said base material at least along a side of said elongate composite structure that is intended to contact the exit penile veins or corpus cavemosa, said coating having better aggressive body fluid resistant properties than said base material.

33. An implantable constriction device according to claim 32, wherein said coating comprises a poly-tetrafluoroethylene (“PTFE”) or poly-para-xylylene polymer coating, or a biocompatible metal coating.

34. An implantable constriction device according to claim 32, wherein said property improving means comprises a core of a viscoelastic material covered with said self-supporting base material.

35. An implantable constriction device according to claim 34, wherein hard silicone constitutes said base material.

36. An implantable constriction device according to claim 34, wherein said viscoelastic material comprises silicone gel, cellulose gel or collagen gel.

37. An implantable constriction device according to claim 32, wherein said base material forms an inflatable tubing.

38. An implantable constriction device according to claim 37, wherein said tubing has an inner surface defining the interior of said tubing, and said coating covers said inner surface.

39. An implantable constriction device according to claim 37, wherein said coating comprises a poly-tetrafluoroethylene (“PTFE”) or poly-para-xylylene polymer coating, or a biocompatible metal coating.

40. An implantable constriction device according to claim 37, wherein hard silicone constitutes said base material.

41. An implantable constriction device according to claim 37, wherein said base material forms two coaxial tubular layers and said property improving means comprises a tubular intermediate layer of a viscoelastic material located between said coaxial tubular layers.

42. An implantable constriction device according to claim 41, wherein said viscoelastic material comprises silicone gel, cellulose gel or collagen gel.

43. An implantable constriction device according to claim 37, wherein said base material forms an outer tubular layer, an inner arcuate layer attached to said outer tubular layer, said outer and inner layers defining a curved space extending longitudinally along said tubing, and said property improving means comprises viscoelastic material filling said space.

44. An implantable constriction device according to claim 43, wherein said viscoelastic material comprises silicone gel, cellulose gel or collagen gel.

45. An implantable constriction device according to claim 31, wherein said property improving means comprises a coating coated on said base material at least along a side of said elongate composite structure that is intended to contact the exit penile veins or corpus cavernosa, said coating having better anti-friction properties than said base material.

46. An implantable constriction device according to claim 45, wherein said coating comprises a poly-tetrafluoroethylene or poly-para-xylylene polymer coating, or a biocompatible metal coating.

47. An implantable constriction device according to claim 45, wherein said property improving means comprises a core of a viscoelastic material covered with said self-supporting base material.

48. An implantable constriction device according to claim 47, wherein hard silicone constitutes said base material.

49. An implantable constriction device according to claim 47, wherein said viscoelastic material comprises silicone gel, cellulose gel or collagen gel.

50. An implantable constriction device according to claim 45, wherein said base material forms an inflatable tubing.

51. An implantable constriction device according to claim 50, wherein said tubing has an inner surface defining the interior of said tubing, and said coating covers said inner surface.

52. An implantable constriction device according to claim 50, wherein said coating comprises a poly-tetrafluoroethylene or poly-para-xylylene polymer coating, or a biocompatible metal coating.

53. An implantable constriction device according to claim 50, wherein hard silicone constitutes said base material.

54. An implantable constriction device according to claim 50, wherein said base material forms two coaxial tubular layers and said property improving means comprises a tubular intermediate layer of a viscoelastic material located between said coaxial tubular layers.

55. An implantable constriction device according to claim 54, wherein said viscoelastic material comprises silicone gel, cellulose gel or collagen gel.

56. An implantable constriction device according to claim 50, wherein said base material forms an outer tubular layer, an inner arcuate layer attached to said outer tubular layer, said outer and inner layers defining a curved space extending longitudinally along said tubing, and said property improving means comprises a viscoelastic material filling said space.

57. An implantable constriction device according to claim 56, wherein said viscoelastic material comprises silicone gel, cellulose gel or collagen gel.

58. An implantable constriction device according to claim 31, wherein said base material forms a first layer and said property improving means comprises a second layer applied on said first layer, said second layer being more fatigue resistant than said first layer.

59. An implantable constriction device according to claim 58, wherein said second layer covers said first layer of said base material along a side of said elongate composite structure that is intended to contact the exit penile veins or corpus cavernosa.

60. An implantable constriction device according to claim 58, wherein said second layer comprises a polyurethane layer.

61. An implantable constriction device according to claim 58, wherein said property improving means comprises a coating coated on said first layer and/or said second layer, said coating having better aggressive body fluid resistance properties and/or better anti-friction properties than said base material.

62. An implantable constriction device according to claim 61, wherein said coating comprises a poly-tetrafluoroethylene or poly-para-xylylene polymer coating, or a biocompatible metal coating.

63. An implantable constriction device according to claim 58, wherein hard silicone constitutes said base material.

64. An implantable constriction device according to claim 58, wherein said first layer of said base material forms an inflatable tubing, and said second layer covers said base material within said tubing.

65. An implantable constriction device according to claim 31, wherein said base material forms an inflatable tubing and said property improving means comprises a liquid impermeable coating coated on said base material.

66. An implantable constriction device according to claim 65, wherein said tubing has an external surface of said base material and an internal surface of said base material defining the interior of said tubing, said coating being coated on said external surface and/or internal surface.

67. An implantable constriction device according to claim 65, wherein said coating comprises a poly-para-xylylene polymer coating, or a biocompatible metal coating.

68. An implantable constriction device according to claim 65, wherein hard silicone constitutes said base material.

69. An implantable constriction device according to claim 65, wherein said base material forms two coaxial tubular layers and said property improving means comprises a tubular intermediate layer of a viscoelastic material located between said coaxial tubular layers.

70. An implantable constriction device according to claim 69, wherein said viscoelastic material comprises silicone gel, cellulose gel or collagen gel.

71. An implantable constriction device according to claim 65, wherein said base material forms an outer tubular layer and an inner arcuate layer attached to said outer tubular layer, said outer and inner layers defining a curved space extending longitudinally along said tubing, and said property improving means comprises viscoelastic material filling said space.

72. An implantable constriction device according to claim 71, wherein said viscoelastic material comprises silicone gel, cellulose gel or collagen gel.

73. An implantable constriction device according to claim 31, wherein said property improving means comprises gas contained in a multiplicity of cavities formed in said base material to improve the flexibility of said composite structure.

74. An implantable constriction device according to claim 73, wherein said cavities are defined by net structures of said base material.

75. An implantable constriction device according to claim 73, wherein Teflon™ constitutes said base material.

76. An implantable constriction device according to claim 73, wherein said composite structure forms an inflatable tubing.

77. An implantable constriction device for constricting penile blood vessels of a patient for treating impotence, the constriction device comprising an elongate composite structure adapted to constrict the exit penile veins or corpus cavernosa of the patient to restrict the penile venous blood flow, wherein the composite structure includes an elongate biocompatible self-supporting base material having surfaces exposed to aggressive body cells, when the constriction device is implanted in the patient, and a cell barrier coating coated on said surfaces to prevent body cells from breaking down the base material.

78. An implantable constriction device according to claim 77, wherein said barrier coating comprises a poly-para-xylylene polymer coating or a biocompatible metal coating.
